(19) United States
(12) Patent Application Publication
(10) Pub. No.: US 2003/0229149 A1
(43) Pub. Date: Dec. 11, 2003

(54) Anti-inflammatory agents

(76) Inventors: Werner Baschong, Basel (CH); Helmut Luther, Grenzach-Wyhlen (DE); Dietmar Ochs, Schopfheim (DE); Sebastien Mongiat, Sierentz (FR)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/444,584

(22) Filed: May 23, 2003

(30) Foreign Application Priority Data
May 28, 2002 (EP) 02405426.4

(51) Int. Cl. A61K 31/075; C07C 43/20

(52) U.S. Cl. 514/721; 568/660

(57) ABSTRACT

Disclosed is the use of hydroxydiphenylether compounds of formula

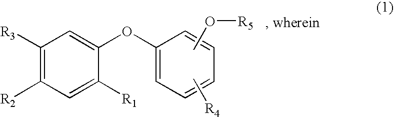

R₁, R₂ and R₃, independently from each other are hydrogen; hydroxy; C₁-C₂₀alkyl; hydroxy-substituted C₁-C₂₀alkyl; C₅-C₇cycloalkyl; C₁-C₂₀alkoxy; C₁-C₆alkylcarbonyl; phenyl; or phenyl-C₁-C₃alkyl;

R₄ is hydrogen, C₁-C₂₀alkyl; hydroxy-substituted C₁-C₂₀alkyl; C₅-C₇cycloalkyl; hydroxy; formyl; acetonyl; allyl; carboxy; carboxy-C₁-C₃alkyl; carboxyallyl; C₂-C₂₀alkenyl; C₁-C₆-alkyl-carbonyl; C₁-C₃alkylcarbonyl-C₁-C₃alkyl; phenyl; or phenyl-C₁-C₃alkyl; and

R₅ is hydrogen; C₁-C₂₀alkoxy; or C₁-C₆alkylcarbonyl;

as pharmaceutical active agents and to pharmaceutical compositions containing them.

The present invention relates to the use of hydroxydiphenylether compounds as pharmaceutical active agents and to pharmaceutical compositions containing them. The invention further relates to these compounds for the preparation of medicaments for the treatment of inflammatory and allergic conditions.

It is standard practice to use glucocorticoids for the topical treatment of inflammatory and allergic conditions. It is common knowledge that these compounds can have unwanted side-effects.

Owing to their insufficient ability to penetrate the skin, nonsteroidal antiinflammatory medicaments containing therapeutic agents such as ketoprofen, BW755c, piroxicam, diclofenac or indomethazin cannot effectively be applied topically, but only systemically (q.v. inter alia G. B. Kasting et al., Pharmacol. Skin., Vol.1, pp. 138-153, Karger, Basel 1987).

It is the object of this invention to provide pharmaceutical compositions having pharmacologically useful properties, in particular antioxidant, anti-inflammatory and antiallergic properties, especially when administered locally.

Surprisingly, it has been found that hydroxydiphenylether compounds of formula

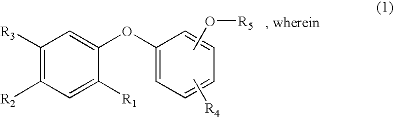

R₁, R₂ and R₃, independently from each other are hydrogen; hydroxy; C₁-C₂₀alkyl; hydroxy-substituted C₁-C₂₀ alkyl; C₅-C₇cycloalkyl; C₁-C₂₀alkoxy; C₁-C₆alkylcarbonyl; phenyl; or phenyl-C₁-C₃alkyl;

R₄ is hydrogen, C₁-C₂₀alkyl; hydroxy-substituted C₁-C₂₀alkyl; C₅-C₇cycloalkyl; hydroxy; formyl; acetonyl; allyl; carboxy; carboxy-C₁-C₃alkyl; carboxyallyl; C₂-C₂₀alkenyl; C₁-C₆-alkyl-carbonyl; C₁-C₃alkylcarbonyl-C₁-C₃alkyl; phenyl; or phenyl-C₁-C₃alkyl; and

R₅ is hydrogen; C₁-C₂₀alkoxy; or C₁-C₆alkylcarbonyl;

exhibit marked antiinflammatory action in cellular and enzymatic in vitro assays and in in vivo assays on human volunteers.

C₁-C₂₀alkyl is straight-chain or branched alkyl radicals such as methyl, ethyl, n-propyl, iso-propyl, n-butyl, sec-butyl, tert-butyl, pentyl, iso-pentyl, tert-pentyl, hexyl, cyclohexyl, heptyl, octyl, isooctyl, nonyl or decyl and the like.

C₁-C₂₀ alkoxy is straight-chain or branched alkoxy radicals such as methoxy, ethoxy, n-propoxy, isopropoxy, n-butoxy, sec-butoxy, tert-butoxy, pentyloxy, iso-pentyloxy, tert-pentyloxy, heptyloxy, octyloxy, isooctyloxy, nonyloxy or decyloxy and the like.

C₁-C₆ alkyl carbonyl is straight-chain or branched carbonyl radicals such as acetyl, propionyl, butyryl, isobutyryl, valeryl, isovaleryl or pivaloyl and the like.

Hydroxy substituted C₁-C₂₀alkyl is hydroxymethyl, hydroxyethyl, hydroxypropyl, hydroxybutyl, hydroxypentyl, hydroxyhexyl, hydroxyheptyl, hydroxyoctyl, hydroxynonyl or hydroxydecyl and the like.

Those compounds of formula (1) have proved particularly interesting in which

R₁, R₂ and R₃, independently of each other, are hydrogen; C₁-C₂₀alkyl; hydroxy-substituted C₁-C₂₀alkyl; or C₁-C₂₀alkoxy;

R₄ is hydrogen; C₁-C₂₀alkyl; hydroxy-substituted C₁-C₂₀alkyl; hydroxy; formyl; acetonyl; allyl; carboxymethyl; or carboxyallyl; and

R₅ is hydrogen; C₁-C₂₀alkoxy; or C₁-C₆alkylcarbonyl;

preferably those, wherein at least one of the substituents R₁, R₂, R₃ and R₄ is not hydrogen.

Compounds of very particular interest are those of formula (1) wherein

R₁ is C₁-C₁₆alkyl; and

R₂, R₃ R₄ and R₅ are hydrogen.

Compounds of very most particular interest are those of formula

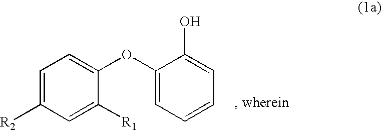

R₁ and R₂ independently of each other are hydrogen; C₁-C₂₀alkyl; or hydroxy-substituted C₁-C₂₀alkyl;

preferably those, wherein at least one of the substituents R₁ and R₂ is not hydrogen.

Furthermore, compounds of formula

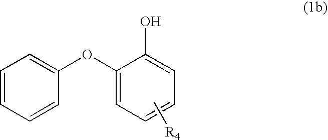

are of particular interest, wherein

R₁ is carboxy; carboxy-C₁-C₃alkyl; C₁-C₃alkylcarbonyl; or C₁-C₃alkylcarbonyl-C₁-C₃-alkyl.

Furthermore, compounds of formula

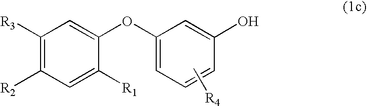

are of particular interest, wherein

R₁, R₂ and R₃ independently from each other are hydrogen; hydroxy; C₁-C₂₀ alkyl; or hydroxy-substituted C₁-C₂₀alkyl; and

R₄ is C,-C₂₀alkyl; hydroxysubstituted C₁-C₂₀; Phenyl; Phenyl-C₁-C₃alkyl; or C₁-C₃alkylcarbonyl;

and most preferably compounds of formula (1c), wherein

R₁, R₂ and R₃ are hydrogen; or C₁-C₂₀alkyl.

Most preferably are compounds of formula (1c), wherein

R₁, R₂ and R₃ are hydrogen; and

R₄ is C₁-C₂₀alkyl; phenyl-C₁-C₃alkyl; or C₁-C₆alkylcarbonyl.

Furthermore, compounds of formula

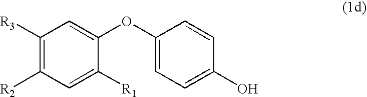

are preferably used, wherein

R₁, R₂, and R₃, independently from each other are hydrogen; C₁-C₂₀alkyl; hydroxy-substituted C₁-C₂₀alkyl; cyclo-C₅-C₇alkyl; phenyl-C₁-C₃alkyl;

Preferably those, wherein at least one of the substituents R₁, R₂, and R is not hydrogen.

Mostly preferred are compounds of formula (1d), wherein

R₁ and R₂, independently of each other are C₁-C₂₀alkyl; most preferably C₁-C₅alkyl; and R₃ is hydrogen.

Further hydroxydiphenylehter compounds, which can be used in the present invention, are listed in the following Table 1:

TABLE 1

| | |
|---|---|
| HD-01 | 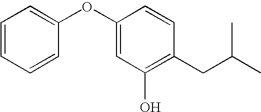 |
| HD-02 | 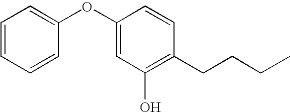 |
| HD-03 | 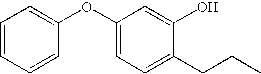 |
| HD-04 | 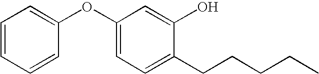 |
| HD-05 | 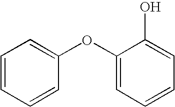 |
| HD-06 | 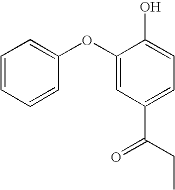 |
| HD-07 | 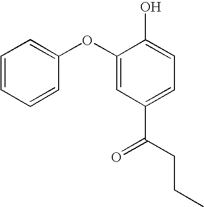 |
| HD-08 | 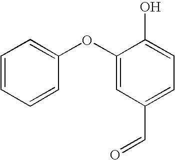 |
| HD-09 | 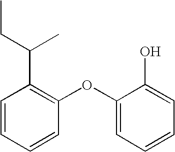 |
| HD-10 | 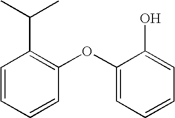 |
| HD-11 | 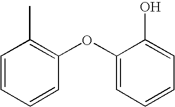 |
| HD-12 | 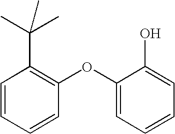 |
| HD-13 | 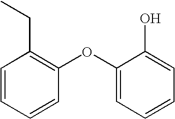 |
| HD-14 | 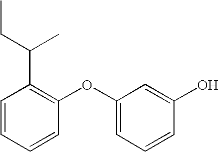 |
| HD-15 | 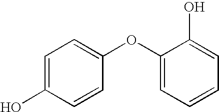 |
| HD-16 | 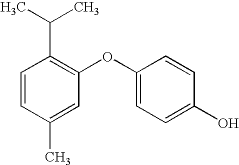 |
| HD-17 | 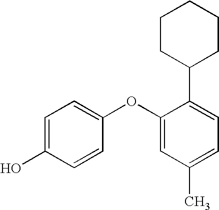 |
| HD-18 | 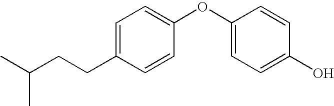 |
| HD-19 | 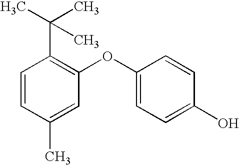 |
| HD-20 | 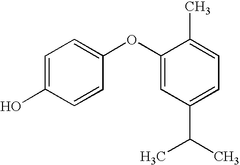 |
| HD-21 | 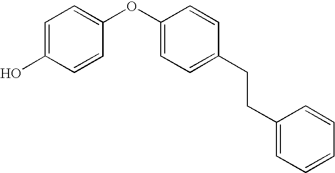 |
| HD-22 | 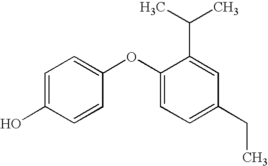 |
| HD-23 | 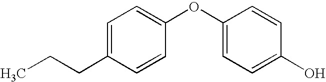 |
| HD-24 | 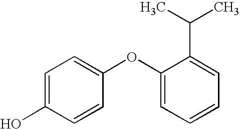 |
| HD-25 | 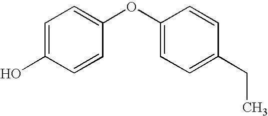 |
| HD-26 | 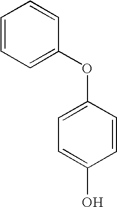 |
| HD-27 | 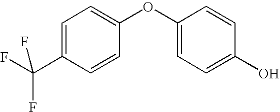 |
| HD-28 | 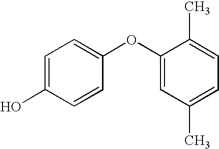 |
| HD-29 | 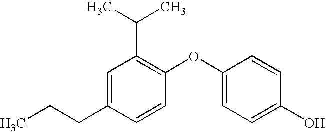 |
| HD-30 | 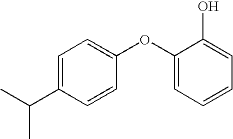 |
| HD-31 | 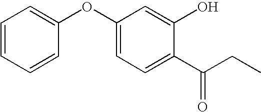 |
| HD-32 | 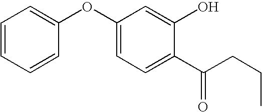 |
| HD-33 | 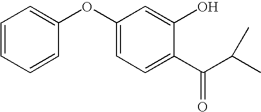 |
| HD-34 | 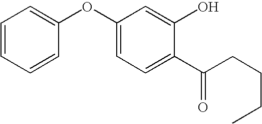 |
| HD-35 | 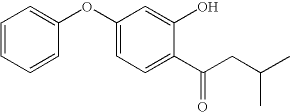 |
| HD-36 | 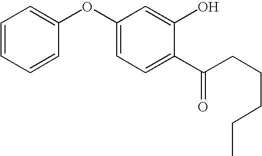 |
| HD-37 | 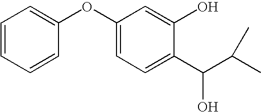 |
| HD-38 | 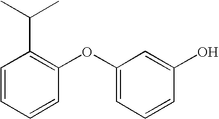 |
| HD-39 | 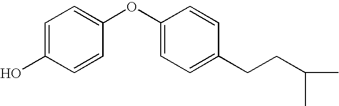 |
| HD-40 | 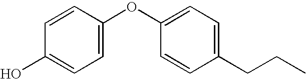 |
| HD-41 | 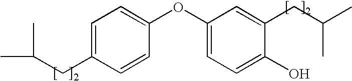 |
| HD-42 | 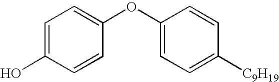 |
| HD-43 | 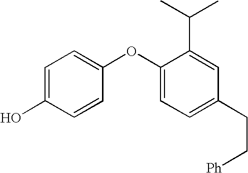 |
| HD-44 | 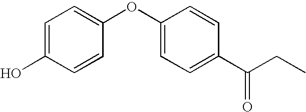 |
| HD-45 | 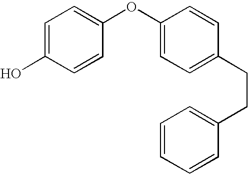 |
| HD-46 | 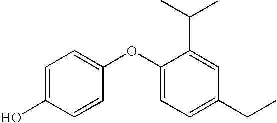 |
| HD-47 | 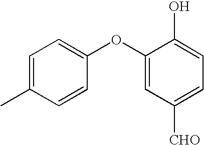 |
| HD-48 | 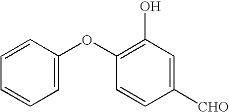 |
| HD-49 | 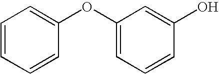 |
| HD-50 | 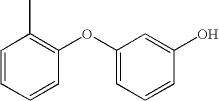 |
| HD-51 | 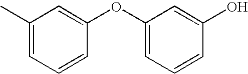 |
| HD-52 | 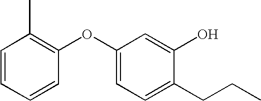 |
| HD-53 | 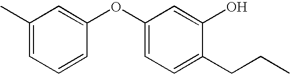 |
| HD-54 | 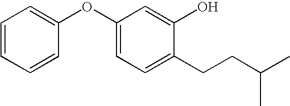 |
| HD-55 | 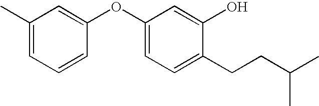 |
| HD-56 | 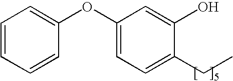 |
| HD-57 | 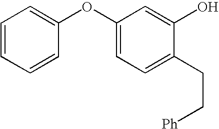 |
| HD-58 | 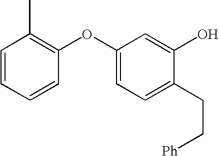 |
| HD-59 | 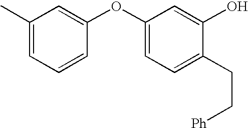 |
| HD-60 | 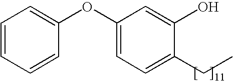 |
| HD-61 | 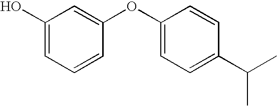 |
| HD-62 | 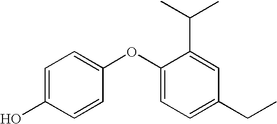 |
| HD-63 | 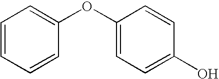 |
| HD-64 | 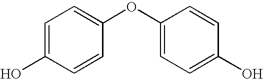 |
| HD-65 | 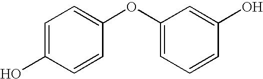 |
| HD-66 | 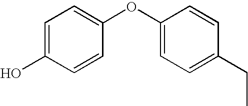 |

The compounds of formula (1) are useful for the treatment of inflammatory and allergic conditions, as well as for the treatment of conditions involving disturbances of cell proliferation.

In vitro assays show that the compounds of formula (1) inhibit the formation of different mediators that are an important factor in inflammation.

The compounds of formula (1) take effect as radical inhibitor. They represent lipoxygenase/-cyclooxigenase inhibitors, i.e. they can intervene in the inflammatory cascade. It can be shown that they take effect as anti-inflammatory agent for the UV induced erythema. They show anti-inflammatory and antioxidant effect comparable to vitamin E.

A further subject of the present invention is a pharmaceutical composition comprising at least one compound of formula (1), together with a pharmaceutically acceptable carrier or adjuvant.

The pharmaceutical compositions are preferably liquid, semisolid or solid preparations.

Examples of liquid pharmaceutical compositions are injectable solutions, infusion solutions, drops, sprays, aerosols, emulsions, lotions, suspensions, drinking solutions, gargles and inhalants.

Examples of semisolid pharmaceutical compositions are ointments, creams (O/W emulsions), rich creams (WIO emulsions), gels, lotions, foams, pastes, suspensions, ovula, plasters, including transdermal systems.

Examples of solid pharmaceutical compositions are tablets, coated tablets, capsules, granules, effervescent granules, effervescent tablets, lozenges, sucking and chewing tablets, suppositories, implants, lyophilisates, adsorbates or powders.

Galenic pharmaceutical compositions comprising the compounds of formula (1) will be understood as meaning in particular emulsions, ointments, gels, sprays and powders.

Compounds of formula (1) may also be contained in liposomes or used in pharmacological compositions with conventional carriers and penetration enhancers, for example urea, dextrane, propylene glycol, oleic acid and the like.

The pharmaceutical composition will usually contain the compounds of formula (1) in amounts of 0.001 to 10% by weight, preferably of 0.1 to 5% by weight, of the total mixture. For the treatment of the conditions listed hereinabove, the pharmaceutical composition of this invention may contain, in addition to the compounds of formula (1), further pharmaceutical agents having antiphlogistic activity, typically including antiinflammatory agents, antipsoriatic agents, cell proliferation regulators, and antiallergic, gastroprotective and antiasthmatic agents.

The following Examples will serve to illustrate the invention without implying any restriction to what is described therein. Unless otherwise indicated, percentages are by weight.

### EXAMPLE 1

### Evaluation of the Efficacy of Hydroxydiphenylether Compounds Against Photooxidative Stress in the Skin Induced by UVA Irradiation

| | | | | Formulation | Formulation |
|---|---|---|---|---|---|
| | Trade Name | INCl-Name | Placebo | 1A | 1A |
| Part A | Cutina GMS | Glyceryl stearate | 4.50 | 4.50 | |
| | Nexbase 2006 FG | Hydrogenated Polydecene | 5.00 | 5.00 | 5.00 |
| Part B | Water Deionized | Aqua | qs 100 | qs 100 | qs 100 |
| Part C | Compound of | | | 0.10 | 0.5 |
| | formula (101) | | | | |
| | Propylene Glycol | Propylene Glycol | 5.00 | 5.00 | 5.00 |
| Part D | Caramel Color | | 0.02 | 0.02 | 0.02 |
| | NaOH 10% | Sodium Hydroxide | qs | qs | Qs |
| | Phenonip | Phenoxyethanol(and) | 0.60 | 0.60 | 0.60 |
| | | Methylparaben(and)Ethyl- | | | |
| | | paraben(and)Butylpara- | | | |
| | | ben(and)Propylparaben | | | |
| | | (and)Isobutylparaben | | | |
| | Salcare SC96 | Polyquaternium-37, Pro- | 0.80 | 0.80 | 0.80 |
| | | pylene Glycol Dicaprylate, | | | |
| | | PPG-1 Trideceth-6 | | | |

Formula (101):

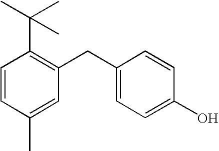

Preparation Method:

The compound of formula (101) is dispersed in propylene glycol at 75° C.

Phase B is heated at 75° C. Before the emulsification, phase C is poured into phase A. Phase A is then poured into phase B under moderate stirring.

The mixture is homogenised with an Ultra Turrax for 10s.

Then the mixture is cooled down to 69° C. with a very moderate stirring.

Salcare SC96 is incorporated into the emulsion and Phenonip added under stirring. Stirring is continued till 40° C., the emulsion colored and pH-adjusted afterwards.

| | |
|---|---|
| Persons tested: | 10 individuals (4 female, 6 male); age range: 28 to |
| | 50 years |
| Body region tested: | back |
| Application phase: | twice a day |
| Test period | 7 days |
| Evaluation method: | Determination of squalene |
| | Determination of squalenehydroperoxide |
| Time of Evaluation: | after UVA radiation |

Descriptive statistics: average, median, minimum, maximum, variance, standard error, standard deviation; Multiple range test.

Test Method

On the back of each subject symmetrically opposed areas are defined. The different formulations are applied twice a day at a dose of about 2 mg/cm² for one week (application with a syringe for fine dosage: Omnifix®-F 1 ml; manufacturer: Braun Melsungen AG, Germany).

Two areas remain untreated.

The unique application of a solution of tocopherol in ethanol (0.2%) before irradiation serves as control.

Use of other cosmetic products is restricted on the test areas throughout the whole study. The areas (exception: one of the two untreated areas was not irradiated and can be attributed to environmental UVA radiation during the test) of the subject's back were then irradiated with UVA light (10 joule/cm²). The lipids present on the test areas are harvested via a solvent extraction (4 ml ethanol for 2 minutes). The samples are first filtered through hydrophobic polypropylene filters to decant squames and other insoluble material, then dried under nitrogen at room temperature and taken up in 1 ml ethanol. Squalene (SQ) and squalenehydroperoxide (SQOOH) are then analysed by High Performance Liquid Chromatography (HPLC).

The results are expressed as the rate of inhibition relative to the untreated area:

% inhibition=100×[SQOOH(untreated)−SQOOH(active)]/SQOOH(untreated)

(SQOOH in pmoles hydrogen peroxide per pg squalene)

HPLC Analysis for SQ

column: LiChrospher® 100 RP-18 (5 μm, 125×4 mm) Merck-Germany mobile phase: acetonitrile/isopropanol (1/1; VN)

detection: UV 210 nm

flow rate: 1 ml/min

injection volume: 20 μl

equipment: Beckman System Gold (USA) with Programmable Solvent Module 126 and Programmable Detector Module 166

HPLC analysis for SQOOH

| | |
|---|---|
| column: | LiChrospher ® 100 RP-Select B (5 μm, 125 × 4 mm) |
| | Merck-Germany |
| mobile phase: | methanol |
| detection: | Chemiluminescence (post column detection) |
| flow rate: | 1 ml/min |
| injection volume: | 20 μl |
| reaction solution: | Luminol (1 μg/ml) and Cytochrom C (10 μg/ml solved |
| | in 50 mM Borate-buffer, pH 10) |
| equipment: | Beckman System Gold (USA) with Programmable |
| | Solvent Module 126 and fluorometer RF-551 |
| | (Shimadzu, Japan) |

The fluorometer is used as a photon detector with the excitation source turned off.

This assay measures the hydroperoxy groups themselves and not indirect indices of lipid peroxidation such as conjugated dienes or breakdown products of lipid hydroperoxides. Chemiluminescence also detects ubiquinols. To confirm that any chemiluminescence observed in this assay was due to a hydroperoxide, not a hydroquinone, some samples were reduced with triphenyl phosphine and rerun. Since the chemiluminescence response of hydroperoxides, but not of hydroquinones, is eliminated by triphenyl phosphine, the disappearance of chemiluminescence peaks in the treated samples indicated that chemiluminescence observed in this assay was due to hydroperoxides and not hydroquinones.

Biometry

Measuring data are centrally computerised after validity check and quality assurance. Evaluation is done using the software Statgraphics for Windows, Version 5.0—Manugistics, USA. Data are analysed by multiple range test (LSD: Least Significant Differences). The 0.05 level is selected as the point of minimal acceptance statistical significance.

Results

The skincare formulations 1A and 1B prevented squalene peroxidation.

In the untreated, non-irradiated area the formation of ethanol extractable squalenehydroperoxide is not detected.

The greatest efficacy has formulation 1B.

The average inhibition in % of peroxidation was in the order of:

| Formulation | Inhibition |
|---|---|
| Placebo | 7 ± 1 |
| Formulation 1A | 13 ± 5 |
| Formulation 1B | 25 ± 7 |
| Control | 24 ± 4 |

Statistically significant differences between the two formulations, the control and the untreated area ccan be detected:

| Formulation | Placebo | 1A | 1B | Control | Untreated |
|---|---|---|---|---|---|
| Placebo | — | s. | s. | s. | s. |
| 1A | s. | — | s. | | s. |
| lB | s. | s. | — | | s. |
| Control | s. | s. | n.s. | — | s. |
| Untreated | s. | s. | s. | | — |

The Placebo shows only a minor efficacy against UVA induced squalene peroxidation. In the untreated area squalene peroxidation increased tremendously.

### CONCLUSION

Pretreatment of the epidermal surface before exposure to UVA irradiation with the formulations according to the present invention prevents an increase in sebum peroxidation. These studies provide compelling evidence for the free radical hypothesis of UVA light induced cutaneous pathology lipid peroxidation increased on irradiation and the topical application with an antioxidant system prevented this damage.

B. Preparation of Pharmaceutical Compositions

### EXAMPLE 2

An ointment containing 2% of the compound of formula (101) can be prepared as follows:

Composition:

| | |
|---|---|
| active ingredient | 2% |
| vaseline | 45.0% |
| paraffin oil | 19.6% |
| cetyl alcohol | 5.00% |
| beeswax | 5.00% |
| sorbitan sesquioleate | 5.00% |
| p-hydroxybenzoate | 0.20% |
| demineralised water to make up | 100.00% |

The fatty substances and emulsifiers are fused together and the active ingredient is dissolved therein. The preservative is dissolved in water and the solution is emulsified at elevated temperature into the melt and the emulsion is then stirred until cold.

### EXAMPLE 3

A cream containing 1% of the compound of formula (101) can be prepared as follows:

Composition:

| | |
|---|---|
| active ingredient | 1% |
| isopropyl palmitate | 8.0% |
| cetyl palmitate | 1.5% |
| silicone oil 100 | 0.5% |
| sorbitan monostearate | 3.0% |
| polysorbate 60 | 3.5% |
| 1,2-propylene glycol PH | 20.0% |
| acrylic acid polymer | 0.5% |
| triethanolamine | 0.7% |
| demineralised water to make up | 100.00% |

The acrylic acid polymer is suspended in a mixture of demineralised water and 1,2-propylene glycol. With stirring, triethanolamine is added to form a mucilage. A mixture of isopropyl palmitate, cetyl palmitate, silicone oil, sorbitan monostearate and polysorbate is heated to ca. 75° C. and the active igredient is dissolved therein. This fatty phase is stirred into the mucilage, which is also heated to c.75° C., and the batch is stirred until cold.

### EXAMPLE 4

A cream containing 0.5% of the compound of formula (101) can be prepared as follows:

Composition:

| | |
|---|---|
| active ingredient | 0.5% |
| cetyl palmitate PH | 2.00% |
| cetyl alcohol PH | 2.00% |
| triglyceride mixture of saturated medium fatty acids | 5.00% |
| stearic acid | 3.00% |
| glycerol stearate PH | 4.00% |
| Cetomacrogol 1000 | 1.00% |
| microcrystalline cellulose | 0.50% |
| 1,2-propylene glycol, dist. | 20.00 vf |
| demineralised water to make up | 100.00%. |

Cetyl alcohol, cetyl palmitate, the triglyceride mixture, stearic acid and glycerol stearate are fused together and the active ingredient is dissolved therein. The microcrystal line cellulose is dispersed in some of the water. Cetomacro gol is dissolved in the remainder of the water and the propylene glycol and the mucilage are mixed therewith. The fatty phase is then stirred into the aqueous phase and stirred until cold.

The formulation is suitable for use as a moisturising skin-protective cream.

### EXAMPLE 5

A transparent hydrogel containing 0.2% of the compound of formula (101) is prepared as follows:

Composition:

| | |
|---|---|
| active ingredient | 0.2% |
| propylene glycol | 10.0-20.0% |
| isopropanol | 20.0% |
| hydroxypropylmethyl cellulose | 2.0% |
| water to make up | 100.00% |

The hydroxypropylmethyl cellulose is swollen in water. The active ingredient is dissolved in a mixture of isopropanol and propylene glycol. Then the active ingredient solution is mixed with a swollen cellulose derivative and, if desired, perfume is added (0.1%).

The formulation is suitable for use as a moisturising skin gel.

### EXAMPLE 6

A transparent hydrogel containing 0.02% of the compound of formula (101) is prepared as follows:

Composition:

| | |
|---|---|
| active ingredient | 0.02% |
| propylene glycol | 20.0% |
| isopropanol | 20.0% |
| acrylic acid polymer | 2.0% |
| triethanolamine | 3.0% |
| water | to make up 100.00% |

Acrylic acid polymer and water are dispersed and the dispersion is neutralised with triethanolamine. The active ingredient is dissolved in a mixture of isopropanol and propylene glycol. The active ingredient solution is then mixed with a gel and, if desired, perfume oil (0.1%) can be added.

### EXAMPLE 7

A foam spray containing 1% of the compound of formula (101) can be prepared as follows:

Composition:

| | |
|---|---|
| active ingredient | 1% |
| cetyl alcohol PH | 1.70% |
| viscous paraffin oil | 1.00% |
| isopropyl myristate | 2.00% |
| Cetomacrogol 1000 | 2.40% |
| sorbitan monostearate | 1.50% |
| 1,2-propylene glycol PH | 5.00% |
| methyl parabene | 0.18% |
| propyl parabene | 0.02% |
| Chemoderm 314 | 0.10% |
| demineralised water to make up | 100.00% |

Cetyl alcohol, paraffin oil, isopropyl myristate, Cetomacrogol and sorbitan stearate are fused together and the active ingredient is dissolved therein. Methyl and propyl parabene are dissolved in propylene glycol and the solution is added to the hot water. The melt and the solution are afterwards mixed. After cooling, the Chemoderm is added and, if desired, perfume oil (0.1%) the formulation is bulked with water to the final weight.

Filling:

An aluminium dispenser is filled with 20 ml of the mixture. The dispenser is fitted with a nozzle and propellant gas is introduced under pressure.

### EXAMPLE 8

An eye ointment containing 0.01% of the compound of formula (101) can be prepared as follows:

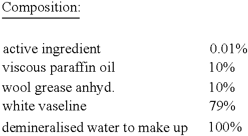

The constituents are fused together and filtered under sterile conditions.

### EXAMPLE 9

Capsules suitable for insufflation and containing 0.025% of the compound of formula (101) can be prepared as follows:

Composition: (for 1000 capsules):

| | |
|---|---|
| active ingredient | 25.00 g |
| milled lactose | 25.00 g |

The active ingredient and the microfine lactose are thoroughly blended. The powder is sieved and filled in 0.05 g portions into gelatine capsules.

What is claimed is:

1. 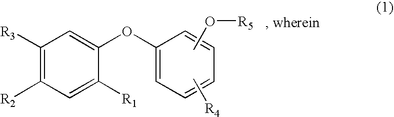
R₁, R₂ and R₃, independently from each other are hydrogen; hydroxy; C₁-C₂₀alkyl; hydroxy-substituted C₁-C₂₀ alkyl; C₅-C₇cycloalkyl; C₁-C₂₀alkoxy; C₁-C₆alkylcarbonyl; phenyl; or phenyl-C₁-C₃alkyl;
R₄ is hydrogen, C₁-C₂₀alkyl; hydroxy-substituted C₁-C₂₀alkyl; C₅-C₇cycloalkyl; hydroxy; formyl; acetonyl; allyl; carboxy; carboxy-C₁-C₃alkyl; carboxyallyl; C₂-C₂₀alkenyl; C₁-C₆-alkyl-carbonyl; C₁-C₃alkylcarbonyl-C₁-C₃alkyl; phenyl; or phenyl-C₁-C₃alkyl; and
R₅ is hydrogen; C₁-C₂₀alkoxy; or C₁-C₆alkylcarbonyl;
for use as a therapeutic agent.

2. A compound according to claim 1, wherein
R₁, R₂ and R₃, independently of each other, are hydrogen; C₁-C₂₀alkyl; hydroxy-substituted C₁-C₂₀alkyl; or C₁-C₂₀alkoxy;
R₄ is hydrogen; C₁-C₂₀alkyl; hydroxy-substituted C₁-C₂₀alkyl; hydroxy; formyl; acetonyl; allyl; carboxymethyl; or carboxyallyl; and
R₅ is hydrogen; C₁-C₂₀alkoxy; or C₁-C₆alkylcarbonyl.

3. A compound according to claim 1 or 2, wherein
R₁ is C₁-C₁₆alkyl; and
R₂, R₃ R₄ and R₅ are hydrogen.

4. A compound according to claim 1, which corresponds to formula 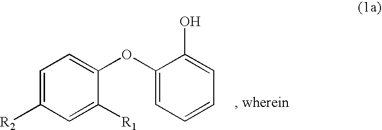
R₁ and R₂ independently of each other are hydrogen; C₁-C₂₀alkyl; or hydroxy-substituted C₁-C₂₀alky.

5. A compound according to claim 1, which corresponds to formula 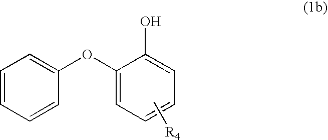
are of particular interest, wherein
R, is carboxy; carboxy-C₁-C₃alkyl; C₁-C₃alkylcarbonyl; or C₁-C₃alkylcarbonyl-C₁-C₃alkyl.

6. A compound according to claim 1, which corresponds to formula 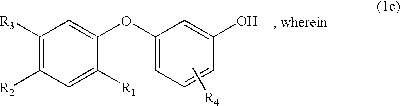
R₁, R₂ and R₃ independently from each other are hydrogen; hydroxy; C₁-C₂₀alkyl; or hydroxy-substituted C₁-C₂₀alkyl; and
R₄ is C₁-C₂₀alkyl; hydroxysubstituted C₁-C₂₀; Phenyl; Phenyl-C₁-C₃alkyl; or C₁-C₃alkylcarbonyl;

7. A compound according to claim 6, wherein R₁, R₂ and R₃ are hydrogen; or C₁-C₂₀alkyl.

8. A compound according to claim 6 or 7, wherein
R₁, R₂ and R₃ are hydrogen; and
R₄ is C₁-C₂₀alkyl; phenyl-C₁-C₃alkyl; or C₁-C₆alkylcarbonyl.

9. A compound according to claim 1, which corresponds to formula 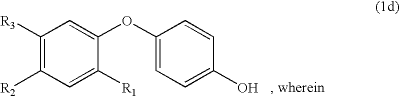
R₁, R₂, and R₃, independently from each other are hydrogen; C₁-C₂₀alkyl; hydroxy-substituted C₁-C₂₀alkyl; cyclo-C₅-C₇alkyl; phenyl-C₁-C₃alkyl.

10. A compound according to claim 9, wherein
R₁ and R₂, independently of each other are C₁-C₂₀alkyl; and
R₃ is hydrogen.

11. A pharmaceutical composition comprising at least one compound as claimed in any one of claims 1 to 10, together with a pharmaceutically acceptable carrier or adjuvant.

12. A pharmaceutical composition according to claim 11, wherein the compound of formula (1) is present in amounts of 0.001 to 10% by weight, of the total mixture.

13. A pharmaceutical composition according to claim 11 or 12, which additionally comprises at least one substance having antiphlogistic action.

14. Use of a pharmaceutical composition according to one of claims 11, 12 or 13 for the local treatment of inflammatory and allergic conditions.

15. Use of a compound according to any one of claims 1 to 10 for the preparation of a medicament for the treatment of radical-induced skin damage and inflammatory and allergic conditions, as well as for the treatment of conditions involving disturbances of cell proliferation.
